(19) United States
(12) Patent Application Publication
(10) Pub. No.: US 2014/0038257 A1
(43) Pub. Date: Feb. 6, 2014

(54) METHODS OF USING ULTRASOUND IN TISSUE CULTURE AND TISSUE ENGINEERING

(71) Applicants: Anuradha Subramanian, Lincoln, NE (US); Joseph Turner, Lincoln, NE (US)

(72) Inventors: Anuradha Subramanian, Lincoln, NE (US); Joseph Turner, Lincoln, NE (US)

(21) Appl. No.: 13/956,742

(22) Filed: Aug. 1, 2013

Related U.S. Application Data
(60) Provisional application No. 61/678,179, filed on Aug. 1, 2012.

(51) Int. Cl. C12N 13/00

(52) U.S. Cl. CPC C12N 13/00
USPC 435/173.8; 435/289.1

(57) ABSTRACT

The present disclosure describes methods of using ultrasound in tissue culture and tissue engineering, as well as a bioreactor that includes at least one ultrasound transducer.

### CROSS REFERENCE TO RELATED APPLICATIONS

This application claims the benefit of priority under 35 U.S.C. 119(e) to U.S. Application No. 61/678,179 filed on Aug. 1, 2012. The prior application is incorporated herein in its entirety.

### FEDERALLY SPONSORED RESEARCH OR DEVELOPMENT

This invention was made with government support under Grant No. R21 RR024437-01A1 awarded by the National Institutes of Health, Department of Health and Human Services. The government has certain rights in the invention.

### TECHNICAL FIELD

This disclosure generally relates to tissue engineering and, more specifically, methods of using ultrasound in tissue engineering.

### BACKGROUND

Bioreactors offer several advantages for culturing cells and tissues compared with simple tissue-flask and Petri-dish culture systems, notably, the ability to provide mechanical forces influencing tissue development and to achieve better control over culture conditions. Designs of bioreactors that are currently available for cultivating tissue-engineered constructs are based primarily on hydrostatic pressure (e.g., dynamic compression), hydrodynamic stress at low shear rates (e.g., perfusion systems), rotating bioreactors, wavy-wall bioreactors, and conventional spinning flasks. Central to a successful tissue engineering strategy to grow functional tissue equivalents is the establishment of a bioreactor or a bioprocessing unit that maintains cells seeded on biodegradable scaffolds and provides essential gas and nutrient transport between the cells and the culture media, as well as the mechanical stimuli necessary to promote extracellular matrix synthesis.

### SUMMARY

The present disclosure describes methods of using ultrasound in tissue culture and tissue engineering, as well as a bioreactor that includes at least one ultrasound transducer.

In one aspect, a method of culturing cells or tissue is provided. Such a method typically includes exposing cells or tissue in culture to intermittent low-intensity-diffuse ultrasound.

In some embodiments, the intermittent low-intensity-diffuse ultrasound includes a frequency of from about 1 MHz to about 8 MHz. In some embodiments, the intermittent low-intensity-diffuse ultrasound includes a pressure of from about 14 kPa to about 60 kPa. In some embodiments, the intermittent low-density-diffuse ultrasound includes a duration of exposure of from about 0.5 min to about 10 mins. In some embodiments, intermittent low-density-diffuse ultrasound includes exposure at an interval of from about 8 times per day up to about 16 times per day.

Representative cells that can be used in the methods described herein include, without limitation, osteoblasts, osteoclasts, osteocytes, chondrocytes, hepatocytes, islet cells, myocytes, epithelial cells, kidney cells, neurons and stem cells. Representative tissue that can be used in the methods described herein include, without limitation, bone, cartilage, liver, pancreas, muscle, epithelium, kidney, uterus, ovarian, and testes.

In another aspect, a method of inducing phosphorylation in cells by extracellular signal-regulated kinases 1 and 2 (Erk1/2) is provided. Such a method typically includes exposing cells in culture to intermittent low-intensity-diffuse ultrasound.

In still another aspect, a method of inducing reorganization of actin in cells is provided. Such a method typically includes exposing cells in culture to intermittent low-intensity-diffuse ultrasound.

In yet another aspect, a bioreactor for culturing cells or tissue is provided. Such a bioreactor typically includes at least one ultrasonic transducer configured to provide an intermittent low-density-diffuse ultrasound to cells or tissues during culture.

In some embodiments, a tissue culture plate comprising the cells or tissue is in fluid communication with the at least one ultrasonic transducer. In some embodiments, the at least one ultrasonic transducer is mounted within a cavity, which is in communication with a tissue culture plate comprising the cells or tissue.

In some embodiments, the bioreactor includes at least two ultrasonic transducers configured to provide an intermittent low-density-diffuse ultrasound to the cells or tissues during culture. In some embodiments, each of the at least two ultrasonic transducers is configured to deliver different frequencies and/or different pressures of intermittent low-density-diffuse ultrasound to the cells or tissue during culture.

In some embodiments, the ultrasonic bioreactor described herein further includes a positioning stage upon which a tissue culture plate is seated, wherein the positioning stage allows for changing the distance between the at least one ultrasound transducer and the cells or tissues that are contained within the tissue culture plate. In some embodiments, the ultrasonic bioreactor described herein further includes a microprocessor.

Representative cells that can be cultured in an ultrasonic bioreactor described herein includes, without limitation, osteoblasts, osteoclasts, osteocytes, chondrocytes, hepatocytes, islet cells, myocytes, epithelial cells, kidney cells, neurons, and stem cells. Representative tissues that can be cultured in an ultrasonic bioreactor as described herein include, without limitation, bone, cartilage, liver, pancreas, muscle, epithelium, kidney, uterus, ovarian, and testes.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a schematic of one embodiment of an ultrasonic bioreactor (UBR) described herein. Panel (a) shows that the programmable stage enables the movement of the holder in x-y-z directions and allows multiple plates to be treated. A plate holder retains the 6-well plates with scaffolds above the transducer array. A custom splitter allows manipulation of the ultrasound signal such that all wells have identical pressure profiles. Panel (b) shows that a K-type thermocouple coupled to a Keithley data acquisition module interfaced with a computer was employed to acquire time dependent temperature profiles upon ultrasound exposure.

FIG. 2 are graphs showing the intensity and pressure profiles. Panel (a) is a graph showing that the splitter was initially tuned so that all the channels provided the same pressure amplitude (14 kPa) at low input voltage (2.5 Vpp) and input frequency of 5 MHz. The pressure amplitudes using other input voltages from the six wells also are shown. Panel (b) shows that the spatial variation in average pressure amplitude was measured in the wells for a 2.5 Vpp.

FIG. 3 are images showing the 2D DIGE global protein expression profiling. A quantitative comparative analysis of protein spots was performed using DeCyder ‘in-gel’ or ‘cross-gel’ analysis software. The color of a protein spot is a measure of its relative abundance. The protein expression ratios between different samples groups were generated as an internal standard and were included in all gels. Proteins that met the cut-off requirement in one or more of the comparisons presented were selected for further identification and analyses. Panel A shows the control (day 3) versus ultrasound (60 kPa, day 3, 8 applications/day); Panel B shows the control (day 6) versus ultrasound (60 kPa, day 6, 8 applications/day); and Panel C shows ultrasound (60 kPa, day 3, 16 applications/day) versus ultrasound (14 kPa, day 6, 8 applications/day).

FIG. 4 are imagines showing ultrasound-induced phosphorylation of Erk at threonine 202 and tyrosine 204. Serum-deprived chondrocytes were treated with ultrasound (5 MHz/14 kPa or 5 MHz/60 kPa) for three minutes, and then total cell lysates were collected at 15 after ultrasound treatment. Total cell lysate for the control was also collected from chondrocytes that did not receive ultrasound treatment. Phosphorylation of Erk1/2 at threonine 202 and tyrosine 204, total Erk1/2 and beta-actin loading control were demonstrated by Western blotting. Experiments were also conducted in the presence of ERK inhibitor, PD98059. Data are representative of three independent experiments.

FIG. 5 are images showing actin organization under ultrasound (right image) or not (left image). Chondrocytes were seeded onto cover slips, placed on the bottom of a 6-well plate, transferred to a plate holder in the UBR and ultrasound stimulation (5 MHz; 14 kPa; 51 s; 6×/day) was applied using the programming module. Upon completion of the stimulation, cells were fixed and stained with phalloidin and visualized under a confocal microscope.

FIG. 6 are graphs showing cellular viability (top) and proliferation (bottom). Cultured chondrocytes were released from control and test scaffolds by adding 0.25% trypsin with 0.1% ethylene diamine tetra acetate followed by incubation at 37° C. with 5% CO₂. Medium was added to the trypsinized cells to bring the final volume to 2 mL. The top graph shows that cell viability was determined using a (4,5-dimethyl-thiazol-2-yl)-2,5-diphenyltetrazolium bromide (MTT) assay (* P<0.05). The bottom graph shows that, in a parallel experiment, cell concentration was counted using a hemocytometer. To obtain a basal value, cell counts were determined after seeding and prior to application of ultrasound stimulation.

FIG. 7 are scanning electron microscopy images of chondrocyte-seeded scaffolds stimulated by ultrasound (right image) or not (left image). Scale bar is shown.

FIG. 8 is a graph showing gene expression. A 5.0 MHz ultrasound signal (14 kPa) was applied six times (6×)/day for 51 s/application and the cells were maintained in culture for 10 days. The mRNA levels of indicated genes were measured by qRT-PCR, using specific primers purchased from Applied Biosystems. The GAPDH gene was used as a loading control. Cells from seeded scaffolds that were not subjected to ultrasound stimulation served as controls. Data were normalized to the controls and are reported as the mean of three independent estimations with error bars, where an error bar represents one standard deviation (* P<0.05).

Like reference symbols in the various drawings indicate like elements.

### DETAILED DESCRIPTION

This disclosure describes the ability of ultrasound stimulation to impact the proliferative and biosynthetic activity of cells in culture. Ultrasound applications can be described according to the intensity of the signal:
diagnostic ultrasound typically uses a frequency between about 3 and about 5 MHz at a low intensity (e.g., about 1 to about 50 mW/cm²);
disruptive ultrasound, such as those used in ultrasonic cleaning devices, typically uses a very low frequency (e.g., about 20 to about 60 kHz) at a high intensity (e.g., up to about 8 W/cm²); and
therapeutic ultrasound, used in medicine and physiotherapy, typically uses frequencies between about 1 and about 3 MHz and intensities of about 0.1 to about 2.0 W/cm².

The methods described herein are directed toward exposing cells or tissues in culture to a particular sub-range within the diagnostic ultrasound range referred to as intermittent low-intensity-diffuse ultrasound. Intermittent low-intensity-diffuse ultrasound is characterized by a very long total application time relative to the maximum travel time of the signal across a culture well. Thus, the cells are excited in an incoherent, or diffuse, manner due, at least in part, to the numerous multiple reflections of the ultrasound waves due to the boundaries of the culture well.

The particular intermittent low-intensity-diffuse ultrasound conditions will be dependent upon the particular cells or tissues being treated, the particular culture conditions (e.g., the presence or absence of a solid support in the media), and the desired outcome of the ultrasound exposure. As described herein, under typical culture conditions (e.g., mammalian chondrocytes with or without a scaffold structure (e.g., chitosan)), low-intensity-diffuse ultrasound conditions include a frequency of from about 1 MHz to about 8 MHz (e.g., about 1 MHz to about 5 MHz, about 1 MHz to about 3 MHz, about 2 MHz to about 7 MHz, about 3 MHz to about 6 MHz, about 4 MHz to about 8 MHz, about 5 MHz to about 8 MHz, about 5 MHz to about 7 MHz, about 3 MHz, about 4 MHz, about 5 MHz, or about 6 MHz) and a pressure of from about 14 kPa to about 60 kPa (e.g., about 15 kPa to about 55 kPa, about 15 kPa to about 40 kPa, about 20 kPa to about 50 kPa, about 25 kPa to about 50 kPa, about 30 kPa to about 60 kPa, about 35 kPa to about 55 kPa, about 40 kPa to about 50 kPa, about 45 kPa to about 55 kPa, about 15 kPa to about 25 kPa, about 15 kPa to about 30 kPa, about 20 kPa to about 30 kPa, about 25 kPa to about 40 kPa, about 30 kPa to about 50 kPa, about 35 kPa to about 50 kPa, about 40 kPa to about 60 kPa, about 45 kPa to about 60 kPa, or about 50 kPa to about 60 kPa).

As used herein, an intermittent low-intensity-diffuse ultrasound signal can be delivered for a duration or length of time of from about 0.5 min to about 10 mins (e.g., about 0.5 min to about 5 mins, about 0.5 min to about 2 mins, about 1 min to about 5 mins, about 1 min to about 8 mins, about 2 mins to about 5 mins, about 2 mins to about 8 mins, about 3 mins to about 5 mins, about 3 mins to about 7 mins, about 4 mins to about 9 mins, about 4 mins to about 7 mins, about 5 mins to about 10 mins, about 5 mins to about 8 mins, about 6 mins to about 8 mins, about 6 mins to about 10 mins, about 6 mins to about 9 mins, about 7 mins to about 10 mins, about 7 mins to about 9 mins, or about 8 mins to about 10 mins). In addition, an intermittent low-intensity-diffuse ultrasound signal can be delivered at an interval of from about 1 or 2 times per day (e.g., once every 10 hours, once every 12 hours, once every 18 hours, once every 24 hours) up to about 20 or more times per day (e.g., once every hour, once every 2 hours, once every 6 hours, once every 8 hours, once every 10 hours). In addition, an intermittent low-intensity-diffuse ultrasound signal can be delivered multiple times in an hour (e.g., once every 2 mins, once every 5 mins, once every 10 mins, once every 15 mins, once every 20 mins, once every 30 mins).

The intermittent low-intensity-diffuse ultrasound described herein is not limited to any particular cells or tissues. Simply by way of example, suitable cells include, without limitation, osteoblasts, osteoclasts, osteocytes, chondrocytes, hepatocytes, islet cells, myocytes, epithelial cells, kidney cells, neurons, and stem cells (e.g., embryonic stem cells (ESCs) or induced pluripotent stem cells (iPSCs)), while suitable tissues include, without limitation, bone, cartilage, liver, pancreas, muscle, epithelium, kidney, uterus, ovarian, and testes. Simply by way of example, bone and cartilage often is irreversibly destroyed following traumatic injury or due to chronic illnesses such as arthritis. Since neither bone nor cartilage exhibits significant self-repair, a promising alternative therapy is the transplantation of tissue-engineered bone or cartilage. The intermittent low-intensity-diffuse ultrasound described herein may be particularly useful for cells or tissues having a major or primary structural protein component (e.g., actin, myosin, collagen, elastin).

At the cellular and molecular level, it is shown herein that applying an intermittent low-intensity-diffuse ultrasound to cells triggered a reorganization of actin in those cells and also activated Erk1/2 (extracellular signal-regulated kinases 1 and 2) pathways. It is well known in the art that, when activated, Erk1/2 undergo phosphorylation and are then translocated to the nucleus, where they regulate, via phosphorylation, a number of different transcription factors. It also is well known in the art that phosphorylation of transcription factors by Erk1/2 leads to changes in gene expression that ultimately regulate proliferation, differentiation, cell cycle processes, and survival, as well as many other cell processes. Therefore, administering intermittent low-intensity-diffuse ultrasound to cells or tissues during growth or during particular phases of growth can have a significant effect on many aspects of cellular processes.

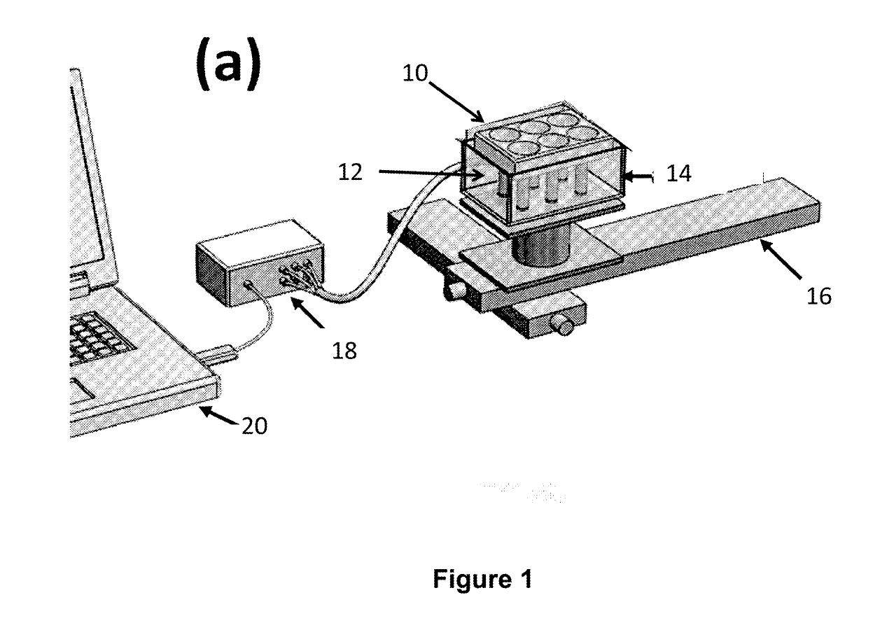

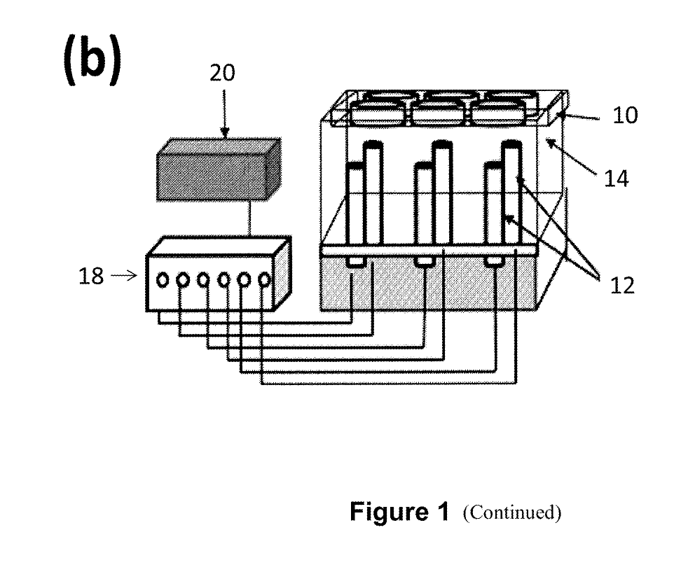

The intermittent low-intensity-diffuse ultrasound described herein can be incorporated into a culture system (e.g., a bioreactor) such that cells or tissues can be exposed to ultrasound in culture. Bioreactors are well known, and generally refer to a device that supports and maintains the viability of cells or tissues in culture and, in some instances, promotes the biological growth and/or development of the cells or tissues. FIG. 1 shows one configuration of certain components of an ultrasonic bioreactor as described herein.

Referring to the exemplary configuration shown in FIGS. 1(a) and 1(b), a tissue culture plate 10 is positioned directly above a cavity 14 containing the ultrasound transducers 12. FIGS. 1(a) and 1(b) show an embodiment having six ultrasound transducers 12, one corresponding to each well in the tissue culture plate 10. It would be understood, however, that any number of ultrasound transducers 12 can be used in an ultrasonic bioreactor as described herein, provided that the transducers 12 deliver the appropriate strength and pressure of signal to the cells or tissue. While FIGS. 1(a) and 1(b) show the ultrasound transducers 12 located within a cavity 14, which helped evenly distribute the ultrasound signal from the transducers, enclosing the transducers 12 within a box 14 is entirely optional, as there may be configurations in which intermittent low-intensity-diffuse ultrasound is delivered to cells or tissue more effectively in the absence of a cavity 14.

An ultrasonic bioreactor also can include a positioning stage 16. In the embodiment shown in FIG. 1(a), the positioning stage 16 is below the box 14 containing the one or more ultrasound transducers 12, such that the box 14 containing the ultrasound transducers 12 can be moved in any of the x-, y- or z-axes relative to the tissue culture plate 10. In some embodiments, however, the positioning stage 16 also could be located above the ultrasound transducers 12 but below the tissue culture plate 10. This configuration would allow for movement of the tissue culture plate in the x-, y- or z-axes relative to the ultrasound transducers 12. In whatever configuration, a positioning stage provides one mechanism by which the distance between the cells or tissue and the ultrasound transducer can be changed, which ultimately provides a mechanism by which the frequency and/or pressure applied to the cells or tissue can be changed. It would be appreciated that the actual position of the ultrasound transducers 12 relative to the tissue culture plates 10 is less relevant than the actual ultrasound signal strength and pressure applied to the cells or tissue.

Also shown in FIGS. 1(a) and 1(b) is a splitter 18 for controlling the signal sent to each transducer 12. FIG. 1(a) shows a computer 20, which, in this particular configuration, provided a power source, which is shown as a simple generator in FIG. 1(b). However, a computer also can be a source of a microprocessor to control the components of the bioreactor, although it would be appreciated that a microprocessor can be provided without the additional components provided by a computer (e.g., screen, keyboard, etc.).

Those skilled in the art would appreciate that at least one ultrasonic transducer can be incorporated into an existing or conventional (e.g., commercially available) bioreactor. Alternatively, a bioreactor can be specifically designed to include, in addition to the other components typically found in a bioreactor, at least one ultrasonic transducer. Those skilled in the art also would appreciate that any configuration of the one or more ultrasound transducers with respect to the cells or tissues in a bioreactor is suitable provided that the culture (i.e., the cells or tissue) can be exposed to the appropriate strength and/or pressure of signal for the appropriate duration and at the appropriate intervals. As described herein, there are certain advantages when the ultrasonic transducer is in fluid communication with the tissue culture plate that contains the cells or tissue or with a structure that holds or supports the tissue culture plate (e.g., a positioning stage). In some embodiments, one or more ultrasonic transducer can be positioned within a fluid-filled structure or cavity that is in contact with or in communication with the tissue culture plate or a structure holding or supporting the tissue culture plate.

It would be understood by those in the art that more than one transducer can be used (e.g., two, three, four, five, six, or more transducers) to expose cells or tissue to ultrasound. More than one transducer can be used to deliver an ultrasound signal to a larger surface area than could be delivered by a single transducer. Additionally or alternatively, more than one transducer can be used to deliver different intermittent low-density-diffuse ultrasound signals to the cells or tissues during culture (e.g., a gradient of signals). For example, different transducers can deliver different frequencies and/or different pressures of ultrasound signal to the culture and/or different transducers can deliver ultrasound signals (e.g., the same or different) for different durations of time and/or different intervals between signals.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Ultrasonic Bioreactor Design

The ultrasonic bioreactor (UBR) was designed to serve as a general research platform for studying cellular response to numerous input parameters in a well-controlled manner. The UBR includes the following components (a schematic is shown in FIG. 1(a)).

Incubator:

All components of the UBR were designed to fit within an off-the-shelf incubator (Form a Model 3033 Steri-Cult incubator) which is used for controlling the temperature, humidity and CO₂ level. The incubator also provided a safe biological condition that minimized possible contamination of the culture plates. This design simplified current and future studies because it allowed utilization of standard six-well TCP plates for the cell growth. A custom insert was designed to hold 9 plates in such a way to allow growth media to be changed periodically and to allow the plates to be excited consistently by the transducer array.

Transducer Array:

An array of six non-focused ultrasonic transducers (Olympus V309, 5 MHz center frequency) was chosen for excitation of the cells within the TCP plates. The transducers were mounted within a fluid-filled cavity to provide consistent water coupling between the transducers and the bottom of the plates. Millipore water was used as the couplant. The transducers were excited using a USB function generator (Tektronix AFG-3021B) for control of the amplitude and frequency of excitation. See, also, FIG. 1(b).

Positioning Stage:

The motion-control assembly consisted of two linear stages for the x-y motion (OWLS-LTM80-300-HSM; maximum horizontal movement of 30 cm) and an elevator stage for the z motion (OWLS-HVM100-30-HSM; maximum vertical movement of 3 cm). The x-y stages move the transducer array to the correct position beneath the TCP plate of interest and the z stage raises and lowers the array during each ultrasound application cycle. These computer-controlled stages provided great flexibility with respect to experiment design—each TCP plate can be excited with a separate ultrasound profile.

Control Software:

Custom control software was written (MATLAB™) for control of the UBR from a laptop computer. This software allows the user to choose the application profile for each TCP plate with respect to ultrasound pressure, frequency, sonication duration, and sonication interval (i.e., number of times per day). The software then determines the application cycle for all plates for each day in order to avoid any timing conflicts. While running, a ‘Sonication Status’ display shows either the plate currently being sonicated or a countdown for the next sonication. At the end of the testing cycle, the software generates a report which serves as a record of all parameters used for the sonications.

### Example 2

### Temperature Effects

A series of experiments (1 to 8 MHz; 14−60 kPa; 1-10 mins of ultrasound exposure) was conducted to investigate the temperature rise induced by the ultrasound regimens employed in the bioreactor, in deionized (DI) water, cell culture medium, within a scaffold in culture medium. A K-type thermocouple coupled to a Keithley data acquisition module was used and interfaced with a computer to acquire time dependent temperature profiles upon ultrasound exposure. Typically, the TCP well was filled with 8 ml of solution and the transducer was fixed at the plate center and in-line with the transducer. Each data point was measured independently in octuplicates.

### Example 3

### Noninertial Cavitation

Noninertial cavitational effects of ultrasound regimens in the bioreactor were assessed through the extent of sucrose hydrolysis in dilute acid and base solutions as detailed elsewhere (Buldakov et al., 2009, Ultrason. Sonochem., 16:392-7). Briefly, 5 ml of sucrose solution (10 mM in 0.1M NaOH or 0.1N HCl) was placed in wells of a 6-well TCP plate, and exposed to ultrasound stimulation. Sucrose hydrolysis in both cases was determined by measuring the glucose content of the samples; by mixing 200 pi of each sample and 1 ml of Glucose (HK) Assay reagent (Sigma) and measuring the absorbance at 340 nm after an incubation at 30° C. for 30 minutes. Glucose concentrations were calculated according to a standard curve established from standard glucose solutions. Sucrose hydrolysis in non-stimulated samples (controls) was measured in a similar manner. Sucrose hydrolysis in 1M HCl served as the positive control. Each data point was measured independently in quadruplicate.

### Example 4

### Inertial Cavitation

Effects from inertial cavitation were examined using electron paramagnetic resonance (EPR). 1-Hydroxy-3-Methoxy Carbonyl 2,2,5,5 tetramethylpyrrolidine hydrochloride (CMH) in Krebs-Henseleit Buffer (KHB) with chelators at pH 7.36 was used as a spin probe for these measurements. 8 ml of degassed DI water was placed in culture wells and 50 μL of CMH (to result in 200 μM solution) was added to the well prior to the indicated ultrasound exposure as detailed earlier. Ultrasound sonication was provided for 8 min, 4 min and 1 minute for each experiment. At the completion of the experiment, 10-20 μL of the solution in the well was pipetted and inserted into glass tubes for measurement of the EPR signal by e-seam BRUKER (NOxygen—Germany). Non-stimulated samples and samples without CMH served as controls. Experiments with a Cup Horn Sonicator 3000 (Ultrasonic Liquid Processor—Misonix) served as the positive control. Each experiment was run independently in quadruplicate.

### Example 5

### Pressure Profiles

The transducer array was designed to deliver a uniform pressure profile within the wells of the TCP plates and to allow a range of pressures to be studied. The pressure profiles were calibrated using a needle hydrophone (Onda HNP 400) as shown in FIG. 1(b). The first measurements were performed to determine the optimum distance between transducer face and the bottom of the TCP plate. This distance is a compromise between the spatial profile of the non-focused transducer and the beam spread that occurs away from it. In addition, the transducer must be close enough to a single well so as not to excite spurious waves within other wells. The hydrophone was placed in the center of a well 8 mm above the bottom plate, a distance representative of cells in a scaffold. The average pressure was measured using six different trials with a 5 MHz excitation frequency. After numerous tests, the transducer array was designed with the transducer face 23 mm below the bottom of the TCP plate, although many other configurations likely would provide an excitation profile sufficient for studying cellular response to ultrasound.

The spatial distribution of the pressure profile was measured in a similar way by mounting the hydrophone to a linear positioning stage. A transducer was placed below one well of a TCP plate at a specified distance. The hydrophone was placed initially at the center of the well 8 mm above the bottom of the plate (see FIG. 1(b)). Pressure measurements were made with an excitation of 5 MHz. The measurements were repeated at numerous positions across the well, using a step size of ˜2 mm. The measured profiles revealed that a region near the well center, ˜1.5 cm wide, has a fairly constant pressure level with a slight drop outside that region. Uncertainty in these measurements is primarily associated with the diffuse interference that occurs as the ultrasound reflects within the well cavity.

### Example 6

### Cell Seeding and Analyses

Discarded bovine shoulder joints from 6-month old calves were obtained from a local abattoir and chondrocytes were isolated using previously described methods (Noriega et al., 2011, Int. J. Carbohydrate Chem., Article ID 809743, pp. 1-13). Freshly isolated chondrocytes were plated on 6-well tissue culture polystyrene plates (TCP) at a seeding density of 2×10⁵ cells/well and maintained in DMEM/F-12 (1:1) (Sigma-Aldrich, St. Louis, Mo.) (3 ml/well) supplemented with 10% FBS (Invitrogen, Carlsbad, Calif.) and 1× antibiotic-antimyotic (Invitrogen). Plates were maintained in the CO₂ incubator for a day. Media was replaced with DMEM/F-12 (3 ml/well) without FBS and incubated again for a day prior to ultrasound exposure. When appropriate, media (DMEM/F-12, w/o FBS) was replaced every 3 days.

### Example 7

### Ultrasound Stimulation

Chondrocytes seeded in six-well tissue culture plates as a monolayer culture were exposed to a low-intensity diffuse ultrasound (LIDUS) signal as listed in Table 1. Cell-seeded scaffolds that are not stimulated served as controls and were handled identically to LIDUS stimulated samples, except for the ultrasound stimulation. Cell-seeded TCP plates were placed in the bioreactor (i.e., plate holders) and, using the programming tool and interface, the unit was programmed to provide dedicated ultrasound stimulation to assigned plates. The ultrasound applications were equally spaced throughout each day of testing. Cell-seeded plates were stimulated and parameters are indicated. Non-stimulated cell-seeded plates served as the control. Three identical but independent experiments were carried out at each condition.

TABLE 1
List of ultrasound parameters used for 2D-DIGE analyses

| Days in culture | kPa | Applications/day | Time per application (mins) |
|---|---|---|---|
| 3 | 60 | 8 | 5 |
| 3 | 60 | 16 | 5 |
| 6 | 14 | 8 | 5 |
| 6 | 60 | 8 | 5 |

### Example 8

### Cell Lysate Preparation

Upon completion of the ultrasound stimulation, plates were removed from the bioreactor; media was pipetted off, washed three times with ice cold PBS (1×) followed by incubation in ice-cold Pierce IP lysis buffer (ThermoScientific Pierce, Rockford, Ill.) supplemented with 1× Halt protease and phosphatase inhibitor cocktail (ThermoScientific Pierce). 200 μL of IP lysis buffer with Halt inhibitor was added to each well and incubated for 15 minutes with periodic mixing at 4° C. prior to combining the lysate from four wells served as replicate of each treatment group. Cell debris was removed and supernatant was collected in new vials after centrifugation at 15,000×g for 10 minutes at 4° C. The protein concentration of each sample was determined using a QuantiPro BCA Assay kit (Sigma-Aldrich). Samples were frozen and shipped to Applied Biomics (Hayward, Calif.) for analyses.

### Example 9

### Preparation of Samples for Two-Dimensional DIGE

Protein samples were resuspended in 2-D cell lysis buffer (30 mM Tris-HCl, pH 8.8, containing 7 M urea, 2 M thiourea and 4% CHAPS). Protein concentration was measured using Bio-Rad protein assay method.

### Example 10

### CyDye Labeling

For each sample, 30 μg of protein were mixed with 1.0 μl of diluted CyDye and kept in the dark on ice for 30 min. The labeling reaction was stopped by adding 1.0 μl of 10 mM Lysine to each sample and incubating in the dark on ice for an additional 15 min. The labeled samples were then mixed together. 2×2-D Sample buffer (8 M urea, 4% CHAPS, 20 mg/ml DTT, 2% pharmalytes and trace amount of bromophenol blue), 100 μl destreak solution and rehydration buffer (7 M urea, 2 M thiourea, 4% CHAPS, 20 mg/ml DTT, 1% pharmalytes and trace amount of bromophenol blue) were added to the labeling mix to bring the total volume to 250 μl. The samples were mixed well and centrifuged before loading into the strip holder.

### Example 11

### IEF and SDS-PAGE

After loading the labeled samples, IEF (pH 3-10 Linear) was run following the protocol provided by GE Healthcare. Upon finishing the IEF, the IPG strips were incubated in freshly made equilibration buffer-1 (50 mM Tris-HCl, pH 8.8, containing 6 M urea, 30% glycerol, 2% SDS, trace amount of bromophenol blue and 10 mg/ml DTT) for 15 minutes with gentle shaking. Then the strips were rinsed in freshly made equilibration buffer-2 (50 mM Tris-HCl, pH 8.8, containing 6 M urea, 30% glycerol, 2% SDS, trace amount of bromophenol blue and 45 mg/ml iodoacetamide) for 10 minutes with gentle shaking. Next, the IPG strips were rinsed in the SDS-gel running buffer before transferring onto 12% SDS-gels. The SDS-gels were run at 15° C. until the dye front ran out of the gels.

### Example 12

### Image Scan and Data Analysis

Gel images were scanned immediately following the SDS-PAGE using Typhoon TRIO (GE Healthcare). The scanned images were then analyzed by Image Quant software (version 6.0, GE Healthcare), followed by in-gel analysis using DeCyder software version 6.5 (GE Healthcare). The fold-change of the protein expression levels was obtained from in-gel DeCyder analysis.

### Example 13

### Protein Identification and Trypsin Digestion

The spots of interest were picked up by Ettan Spot Picker (GE Healthcare) based on the in-gel analysis and spot picking design by DeCyder software. The gel spots were washed a few times then digested in-gel with modified porcine trypsin protease (Promega). The digested tryptic peptides were desalted using a Zip-tip C18 (Millipore). Peptides were eluted from the Zip-tip with 0.5 μl of matrix solution (alpha-cyano-4-hydroxycinnamic acid (5 mg/ml in 50% acetonitrile, 0.1% trifluoroacetic acid, 25 mM ammonium bicarbonate) and spotted on a MALDI plate.

### Example 14

### Mass Spectrometry

MALDI-TOF MS and TOF/TOF tandem MS/MS were performed on AB SCIEX TOF/TOF™ 5800 System (AB SCIEX). MALDI-TOF mass spectra were acquired in reflectron positive ion mode, averaging 4000 laser shots per spectrum. TOF/TOF tandem MS fragmentation spectra were acquired for each sample, averaging 4000 laser shots per fragmentation spectrum on each of the 7-10 most abundant ions present in each sample (excluding trypsin autolytic peptides and other known background ions).

### Example 15

### Database Search

Both of the resulting peptide mass and the associated fragmentation spectra were submitted to GPS Explorer workstation equipped with MASCOT search engine (Matrix science) to search the database of National Center for Biotechnology Information non-redundant (NCBInr). Searches were performed without constraining protein molecular weight or isoelectric point, with variable carbamidomethylation of cysteine and oxidation of methionine residues, and with one missed cleavage also allowed in the search parameters. Candidates with either protein score C.I. greater than 95% were considered significant.

### Example 16

### Scaffold Preparation and Cell Seeding

Chitosan scaffolds (CS) were prepared by the freeze drying and lyophilization method as detailed elsewhere (Hasanova et al., 2011, J. Tissue Eng. Regen. Med., 5:815-22). Briefly, a 2% w/v solution of chitosan (81.7% de-acetylated, MM=276 kDa, Vanson HaloSource, Wash.) in 1% acetic acid was prepared and pipetted into each well of a 24-well tissue culture polystyrene plate (TCP; Falcon Brand, Fisher, Pa.), frozen at −20° C. and then lyophilized for 24 to 36 hours. Scaffolds (5 mm×5 mm) were punched out using a biopsy punch and neutralized with 0.25 M NaOH for 30 minutes, copiously rinsed with deionized water (DI) and sterilized with 70% ethanol solution for 1 hour; rinsed with sterile DI water, followed by sterile phosphate buffer saline (PBS) and then incubated for 12 hours in medium (DMEMIF-12 with 10% FBS) to obtain pre-wetted scaffolds. Pre-wetted scaffold discs were seeded with bovine chondrocytes (passage-2) at a seeding density of 3×10⁴ cells/scaffold, with six scaffolds per well in a 6-well tissue culture plate. One plate with 36 scaffolds represented one test condition. Typically, 15 μl of a 2.0×10⁶ cells/ml stock solution was pipetted on each scaffold, plates were kept in CO₂ incubator at 37° C. and 95% RH for 3 hours and then 8 ml of fresh medium was added on top of the scaffolds and maintained for 20 hours in the incubator. Scaffolds were transferred to a new TCP plate with 5-8 ml of fresh complete media per well and placed in the incubator for 3 days and were then subjected to ultrasound stimulation. Control treatments did not include ultrasound stimulation and were handled similarly to ultrasound-treated specimens. Medium was changed every alternate day. Unseeded disks were also included as controls.

### Example 17

### Cell Proliferation

Cultured chondrocytes were released from control and test scaffolds by adding 0.25% trypsin with 0.1% EDTA (ethylene diamine tetra acetic acid) followed by incubation at 37° C. with 5% CO₂. Medium was added to the trypsinized cells to bring the final volume to 2 mL. Cell concentration was counted using a hemocytometer. To obtain a basal value, cell counts were first determined 3 days after seeding and prior to application of ultrasound stimulation. In a parallel experiment, the cell viability was also determined by (4,5-dimethyl-thiazol-2-yl)-2,5-diphenyltetrazolium bromide (MIT) assay.

### Example 18

### mRNA Gene Expression Analysis

Upon completion of ultrasound exposure, cell-seeded tissue culture plates were washed with ice-cold HBSS, and incubated with 200 μl/well of Trizol reagent (Invitrogen) with periodic mixing for 5 mins and cell homogenate was collected. RNA was isolated from cell homogenate using Qiagen RNeasy mini kit (Qiagen, Valencia, Calif.). The mRNA level was quantification by using quantitative real-time PCR (qRT-PCR). The qRT-PCR analysis was carried out using QuantiFast Probe RT-PCR Kit (Qiagen). 50 ng of total RNA were added per 10 μl reaction vial with RT mix, RT-PCR master mix, sequence-specific primers and Taqman probes. Sequences for all target gene primers and probes were purchased commercially from Applied Biosystmens, Foster City, Calif. (GAPDH was used as an internal control; Applied Biosystems). qRT-PCR assays were carried out in triplicate on Eppendorf s Mastercycler RealPlex Real Time PCR system (Eppendorf North America, Hauppauge, N.Y., USA). The cycling conditions were 10 min cDNA formation by reverse transcriptase enzyme at 50° C. and 5 min polymerase activation at 95° C. followed by 40 cycles at 95° C. for 30 sec, at 55° C. for 30 sec and 72° C. for 1 min. The threshold was set above the non-template control background and within the linear phase of target gene amplification to calculate the cycle number at which the transcript was detected.

### Example 19

### Chondrocyte Cell Morphology on the Samples

For SEM microscopy, cells seeded in scaffolds were crosslinked with 2.5% glutaraldehyde (Sigma) in phosphate buffered saline (PBS) for 30 minutes, rinsed with deionized water, and gradually dehydrated with series of ethanol solutions. Hexamethyl disilazane (Fisher, Pa.) was used to remove 100% ethanol. Samples were sputter coated with Au—Pd before they were examined under SEM (Hitachi, S-3000N variable pressure, Japan). A voltage of 15 kV was used to visualize the samples.

### Example 20

### Quantification Analysis

Band intensities were quantified by densitometry using ImageQuant software (v5.2, Molecular Dynamics). The values reported were normalized to unstimulated controls. For the analysis of the ultrasound stimulation effects on mRNA levels, data represent the mean and standard deviation values of three independent estimations.

### Example 21

### Staining for Actin

To visualize actin organization under ultrasound in the UBR, chondrocytes were seeded onto coverslips at a seeding density of 2×10⁴ cells/coverslip, placed at the bottom of a 6-well TCP plate, filled with 8 ml of media and transferred to the plate holders in the UBR. Ultrasound stimulation was applied using the programming tool/interface. Non-stimulated cells on coverslips served as controls. Cells grown on coverslips were fixed in 4% paraformaldehyde (PFA) (Electron Microscopy Sciences, Pa., USA) in PBS for 2 hours at room temperature followed by washing three times with TBS. Then coverslips were permeated with 0.1% Triton X-100 (Sigma-Aldrich) prepared in TBS for 15 minutes; washed three times with TBS and then blocked for 30 minutes using blocking solution (1% bovine serum albumin in TBS). Subsequently, covers lips were stained with a 1:50 dilution of Alexa-Flour 594 phalloidin in blocking solution for 30 minutes at room temperature and rinsed extensively prior to mounting with aqueous mounting medium on coverslips. A confocal laser scanning microscope (Olympus FV500 Inverted Olympus IX 81) was used to obtain the images. To minimize the autofluorescence, images were converted into black and white images, and were processed using ImageJ™.

### Example 22

### Statistical Analyses

Statistical significance was evaluated using one-way analysis of variance for comparison between the control and test groups. The values were considered to be statistically different when P<0.05.

### Example 23

### The Bioeffects of Ultrasound

The UBR uses ultrasound to stimulate chondrocytes maintained in in vitro culture (FIG. 1) over a range of ultrasound stimulations. Of equal importance to the direct aspects of ultrasound, the UBR must not exhibit any effects that may result from secondary bioeffects. When biological materials are exposed to ultrasound, the associated thermal and non-thermal mechanisms can impact or, in certain cases, act as the causative agent for the observed bioeffects, namely, proliferation, viability and cell-specific processes. Because the focus is to understand the biophysical effects of the adopted ultrasound stimulation regimen, it is critical that the contribution of ultrasound-induced thermal and non-thermal mechanisms on the observed cellular effects be delineated.

Thermal effects, which are typically associated with an increase in the bulk temperature of the medium, are usually exerted at high ultrasound intensities. In these studies, low intensity ultrasound signals were used. The temperature increases (ΔT) caused by exposure to ultrasound stimulation regimens with amplitudes ranging from 10-60 kPa at frequencies of 5.0 to 8.5 MHz, were determined in sterile water, using Dulbecco's Modified Eagle Medium (DMEM), within a scaffold in DMEM and within a cell-seeded scaffold. No detectable temperature rise was observed.

Non-thermal bioeffects can be grouped into two categories: inertial cavitational and noninertial-cavitational mechanisms. Inertial cavitation (IC) generally occurs at higher acoustic pressures and noninertial cavitation (non-IC) occurs at lower acoustic pressures. The threshold for non-IC has been previously reported to be in the range between 6-8 W/cm². Intensities of 0.01-0.1 W/cm² were observed in the bioreactor such that no significant non-IC was expected. However, the non-IC effects of ultrasound also were assessed by measuring the extent of sucrose hydrolysis in aqueous solutions. No discernible levels of hydrolysis were noted in control or solutions exposed to ultrasound, where a wide regimen of ultrasound was tested and appropriate positive controls were included.

To assess possible IC effects, a spinprobe (CMH) was added to the aqueous media at room temperature and the solutions were sonicated over a wide range of ultrasound regimens employed in the bioreactor. The relative amount of electron paramagnetic resonance (EPR) signal (an indicator of the degree of IC) was negligible and was not significantly different from the EPR signal strengths obtained in control samples. However, the use of a sonic-horn (i.e., positive control) resulted in a significantly higher level of EPR signal (5.5-times higher). The ability of the ultrasound regimen to generate reactive oxygen species (ROS) was also evaluated, using the Image-iT™ LIVE Green ROS Detection Kit, where the oxidatively stressed and non-stressed cells are reliably distinguished by fluorescence microscopy. These results suggest that ROS was not generated by the ultrasound stimulation regimen used in this study. It was concluded that any ultrasound effects in the bioreactor, aside from cellular responses, were negligible.

### Example 24

### Characterization of the Ultrasound Field

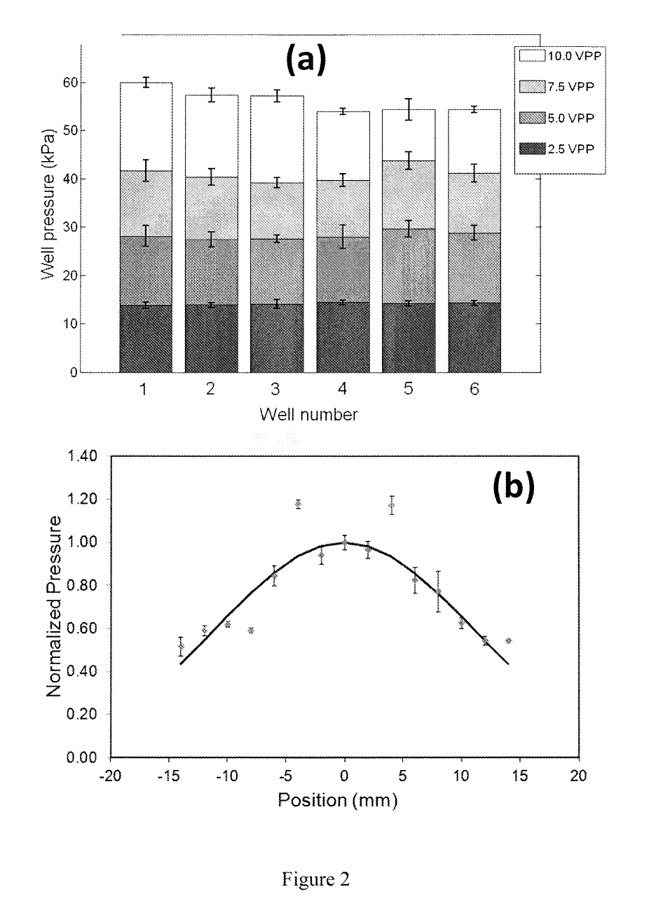

The automated operation of the bioreactor is dependent on (a) the tuning of splitter channels, which, in turn, controls the transducers to provide equal pressure amplitude; (b) placement of the transducers; (c) controlled movement of the x-y-z stage. Thus, the splitter was first tuned for a 5.0 MHz, 14 kPa output for all transducers (values shown to be effective in previous work (Noriega et al., 2007, Tissue Eng., 13:611-8)). The ability of the splitter to provide other inputs, uniformly, also was tested. Finally, the movement of the aquarium in x-y-z direction was controlled to avoid any spillage of water. The variation of pressure amplitude in the wells is shown in FIG. 2(a) with respect to input voltage (Vpp). These measurements were made at points that were 8 mm above the bottom of the TCP plates and along the axis of the transducer. In the bioreactor configuration described herein, numerous multiple reflections of the ultrasound waves are possible from the boundaries of the well and result in a diffuse field. The average radial variation of the pressure amplitude inside a well was also evaluated and is shown in FIG. 2(b). From these results, it was estimated that the usable width within each well was ˜1.5 cm, a width within which the pressure profile is uniform.

While using the bioreactor, wide arrays of ultrasound regimens are possible. In this study, the analyses were confined to a signal frequency (center) of 5 MHz so that conformity could be established with previously reported results and also to be able to perform comparative analyses.

### Example 25

### Ultrasound-Induced Proteome Changes in Chondrocytes

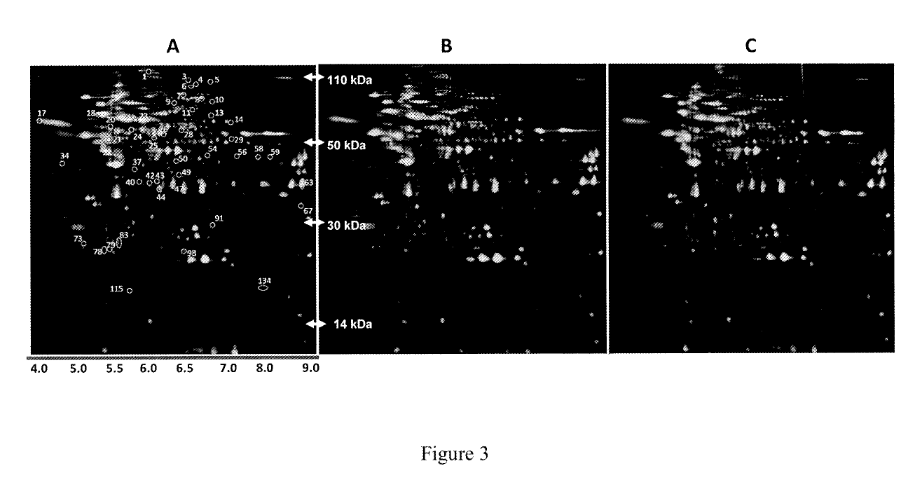

Although the effects of ultrasound on various cell types have been widely studied using DNA microarrays (Tabuchi et al., 2002, Biochem. Biophys. Res. Comm., 290:498-503; Sironen et al., 2002, Biochem. Biophys. Acta, 1591:45-54; Myokai et al., 2003, J. Period. Res., 38:255-61), the effect of ultrasound on chondrocytes has not been investigated comprehensively. We have used a proteomic approach to profile the ultrasound-induced protein expression and modifications. The protein concentration was measured and adjusted so that the same amount of each protein sample was labeled with size and charge-matched minimal fluorescent CyDye and separated on an analytical scale electrophoresis gel. A representative 2D-DIGE image of protein lysates from ultrasound-treated chondrocytes is shown in FIG. 3. About 138 protein spots were resolved and identified with high confidence (>95%). Overall, around 138 protein spots were found consistently up- or down-regulated by over 1.3-fold in triplicate experiments (in 9 different comparative gels) after ultrasound treatment (5 MHz; 5 minutes, 14 to 60 kPa) for 3 or 6 days. An initial effort was made to identify around 50 protein spots, encompassing a wide range of molecular weights, pI values, fold changes, and abundance. All 50 protein spots were identified successfully with high confidence by using preparative gel electrophoresis, in-gel trypsin digestion followed by tandem mass spectrometry as described earlier, and the location of each spot was labeled with a number. Proteins identified to date are listed in Table 2 and are grouped according to their primary functions.

TABLE 2
Proteins Identified in 2S-DIGE gels

| | | | Molecular | | | | |
|---|---|---|---|---|---|---|---|
| Spot | | Protein | mass kDa | pl | | | |
| no: | Protein name | Accession no. | (theoretical) | (theoretical) | Panel-A | Panel-B | Panel-C |
| | Cytoskeletal proteins | | | | | | |
| 13 | Lamin A | gi\|453180 | 71598.4 | 6.20 | 1.22 | −1.42 | 1.20 |
| 21 | Vimentin | gi\|110347570 | 53695.1 | 5.06 | 1.59 | 1.11 | 1.13 |
| 37 | b-Actin | gi\|14250401 | 40978.4 | 5.56 | −1.01 | 1.32 | −1.04 |
| 50 | ACTR1A | gi\|75775168 | 41436.4 | 6.59 | 1.30 | 1.07 | −1.27 |
| | Cell membrane-bound | | | | | | |
| | molecules | | | | | | |
| 1 | Sorbin and SH3 domain | gi\|114597167 | 117336.7 | 8.53 | −1.00 | −2.36 | −1.05 |
| | containing 2 isoform | | | | | | |
| | Proteins involved in matrix | | | | | | |
| | synthesis | | | | | | |
| 24 | prolyl 4-hydroxylase subunit | gi\|115495019 | 60972 | 5.63 | −1.57 | 1.64 | 1.12 |
| | alpha-1 precursor | | | | | | |
| 47 | Annexin | gi\|74 | 38873.2 | 6.44 | −1.02 | −1.04 | −1.42 |
| | Metabolic enzymes | | | | | | |
| 7 | Hexokinase | gi\|60592784 | 102141 | 6.29 | 1.46 | −1.22 | −1.99 |
| 43 | Transaldolase | gi\|164420731 | 37657.6 | 7.03 | 1.24 | 1.33 | −1.11 |
| 11 | Transketolase | gi\|152941228 | 64834.1 | 6.71 | 1.43 | −1.04 | −1.52 |
| 63 | Glyceraldehyde-3-phosphate | gi\|77404273 | 35845.3 | 8.50 | 1.38 | −1.04 | −1.41 |
| | dehydrogenase | | | | | | |
| 49 | Acetyl-CoA acetyltransferase | gi\|115495669 | 41172.2 | 6.46 | 1.32 | 1.13 | −1.64 |
| 44 | I-lactate deydrogenase | gi\|118572666 | 36700.2 | 6.02 | 1.10 | 1.02 | −1.21 |
| | Protein synthesis and | | | | | | |
| | degradation | | | | | | |
| 25 | 26S protease regulatory | gi\|296222266 | 49275.7 | 5.97 | 1.35 | 1.03 | −1.29 |
| | subunit 4-like | | | | | | |
| 98 | Protein DJ-1 | gi\|62751849 | 20022.6 | 6.84 | 1.36 | 1.13 | −1.34 |
| 6 | Elongation factor-2 | gi\|115497900 | 95307.0 | 6.41 | 1.52 | −1.03 | −1.15 |
| 8 | Glutaminyl-tRNA synthetase | gi\|77735887 | 87587.6 | 6.43 | 1.36 | −1.04 | −1.04 |
| 10 | Threonyl-tRNA synthetase, | SYTC_BOVIN | 83438.9 | 6.34 | 1.04 | −2.00 | −1.14 |
| | cytoplasmic | | | | | | |
| 22 | Protein disulfide- | gi\|148230374 | 56893.9 | 6.38 | 1.12 | 1.01 | −1.12 |
| | isomerase A3 precursor | | | | | | |
| 56 | Peptidyl-prolyl cis- | FKB11_BOVIN | 22460.3 | 9.26 | 1.31 | 1.42 | −1.15 |
| | trans isomerase | | | | | | |
| 40 | Chain A, Crystal Structure | gi\|109157318 | 30401.6 | 5.71 | 1.32 | 1.12 | −1.05 |
| | Of Dimethylarginine | | | | | | |
| | Dimethylaminohydrolase I | | | | | | |
| | In Complex With S-Nitroso- | | | | | | |
| | RNA and DNA binding | | | | | | |
| | proteins | | | | | | |
| 42 | serine/threonine-protein | gi\|296482303 | 35208.5 | 5.20 | 1.17 | 1.43 | −1.15 |
| | phosphatase 2A 65 kDa | | | | | | |
| | regulatory subunit A | | | | | | |
| | alpha isoform | | | | | | |
| 73 | translationally- | gi\|62177164 | 19568.6 | 4.84 | −1.34 | 1.06 | 1.22 |
| | controlled tumor protein | | | | | | |
| 9 | ATP-dependent RNA | gi\|115495959 | 82361.8 | 6.81 | 1.37 | −1.01 | −1.11 |
| | helicase DDX | | | | | | |
| 54 | COMM domain containing | COMD7_BOVIN | 22519.6 | 5.69 | 1.30 | 1.11 | −1.03 |
| | protein 7 | | | | | | |
| 3 | RNA polymerase II- | RPAP1_BOVIN | 152762.7 | 5.91 | 1.31 | −1.39 | −1.41 |
| | associated protein 1 | | | | | | |
| 17 | Chain D, Crystal Structure | gi\|306991567 | 50283.3 | 4.96 | −2.46 | −1.70 | 0.79 |
| | Of Bovine F1-C8 Sub- | | | | | | |
| | Complex Of Atp Synthase | | | | | | |
| | Others | | | | | | |
| 18 | 78 kDa glucose-regulated | gi\|115495027 | 72355.5 | 5.07 | −1.33 | 1.43 | −1.12 |
| | protein precursor (HSP-70) | | | | | | |
| 78, 83 | apolipoprotein A-I | gi\|75832056 | 30257.9 | 5.71 | −1.73 | −1.74 | 1.10 |
| | preproprotein | | | | | | |
| 79 | heme-binding protein 1 | gi\|115496135 | 21216.4 | 5.39 | 1.45 | 1.56 | −1.18 |
| 115 | Ferritin, heavy polypeptide | gi\|154426178 | 21056.2 | 5.54 | −1.01 | 1.50 | 1.06 |
| 91 | Hemoglobin subunit alpha-I/II | HBA_BISBO | 15129.9 | 8.90 | 1.35 | 1.33 | 1.30 |
| 29 | MRS2 | gi\|46362574 | 46461.3 | 5.78 | 1.45 | 1.24 | −1.29 |
| 4 | High density lipoprotein | gi\|297473540 | 175738.3 | 9.32 | 1.04 | −2.17 | −1.21 |
| | binding protein | | | | | | |
| 14 | Alpha-2-HS- | gi\|27806751 | 38394.4 | 5.26 | 1.32 | −1.32 | −1.38 |
| | glycoprotein precursor | | | | | | |
| 28 | Reticulocalbin-3 precursor | gi\|114053121 | 37545.1 | 4.76 | 1.30 | −1.02 | −1.25 |
| 34 | cathespin | gi\|299522 | 37686.8 | 5.43 | −1.43 | 1.19 | 1.05 |
| | Molecular chaperones | | | | | | |
| 26 | T-complex protein 1 | gi\|77736031 | 57919.6 | 6.32 | 1.15 | −1.02 | −1.34 |
| | subunit zeta | | | | | | |
| 58, 59 | Serpin HI precursor | gi\|114051505 | 46477.2 | 9.01 | 1.50 | 1.18 | 1.18 |
| | Ion Channels | | | | | | |
| 67 | Plasmalemmal porin | gi\|437027 | 30675.6 | 8.84 | 1.37 | 1.11 | −1.48 |

### Example 26

### Functional Category or Altered Proteins

To gain additional insights into the biological significance and functional attributes of the differentially expressed proteins during ultrasound, the proteins were categorized according to their main biological functions collected from the UniProt protein knowledge database and PubMed. According to their main biological functions, the proteins were classified as follows: energy metabolism, RNA/DNA binding proteins/chromatin assembly, cytoskeletal, matrix synthesis, protein synthesis and degradation, and others.

### Example 27

### Validation of Differentially Expressed Proteins

Ultrasound-Induced Erk1/2 Phosphorylation

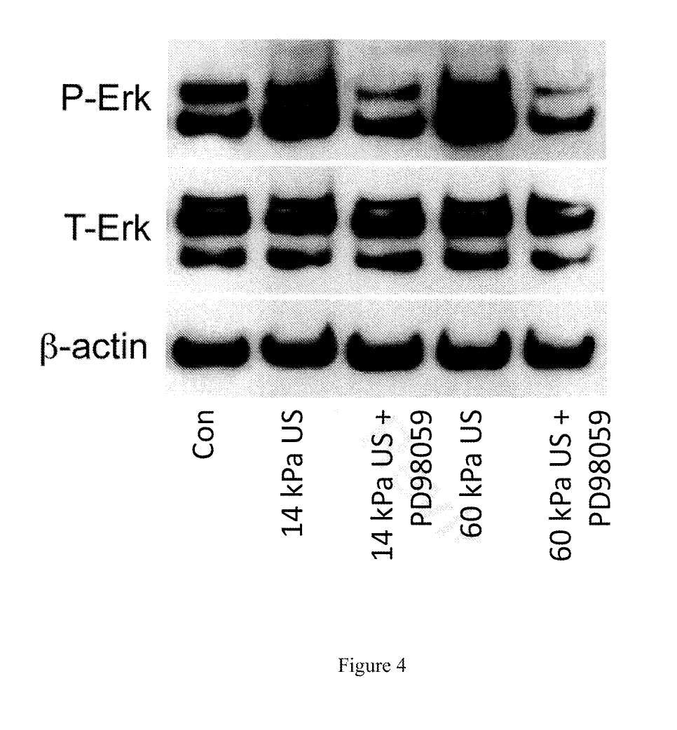

Chondrocytes that were serum-deprived overnight were treated with 5.0 MHz ultrasound (14 kPa) for three minutes, and then the cells were lysed to collect protein 15 minutes after the ultrasound mechanical stress. Western blotting was used to analyze the phosphorylation of Erk1/2 at threonine (T)202/Y204 of Erk1 and T185/Y187 of Erk2 compared to total Erk1/2. Ultrasound stimulation at a central frequency of 5 MHz induced transient phosphorylation of Erk1/2 (FIG. 4); with a greater level of p-ERK1/2 at 10 Vpp (60 kPa) as compared to 2.5 Vpp (14 Kpa). The addition of ERK inhibitor (PD98059) to the medium was observed to reduce the phospho-ERK signals to baseline levels, suggesting a role for ERK-mediated signaling pathway under ultrasound.

### Example 28

### Reorganization of Actin Following Exposure to Ultrasound

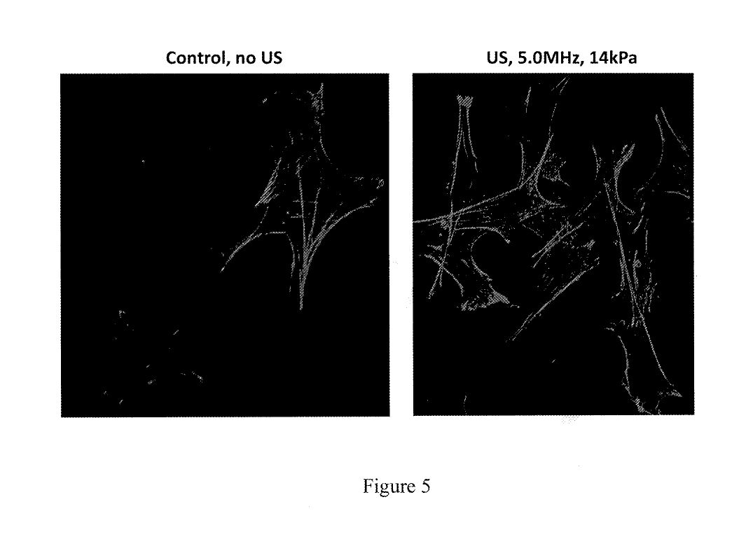

Confocal microscopy was used to assess the distribution and organization of the actin, where determinations were made before and after ultrasound stimulation and are shown in FIG. 5. The left panel is a representative frame showing the actin distribution in control, non-stimulated cells. Several actin fiber formations can be noted in these images, the presence of long actin fibers that run along the length of the cell is evident and an actin mesh that surrounds the cells was also noted. In general, most control cells exhibited this type of actin organization. The right panel shows the distribution of actin in cells that were stimulated by ultrasound. Actin structure appeared to be non-organized, with punctuated membrane, and cytosolic located F-actin. Few long actin filaments were also noted along with mesh like actin structures and long thin processes; however, fewer stress fibers were observed.

A preliminary quantification of the actin cytoskeleton was done using ImageJ™ and a decrease is the total number of actin filaments was noted in ultrasound treated samples (6.6±1.4 actin filaments) when compared with the control samples (21.0±8.0 actin filaments). Also, the ratio of the “Feret diameter/mini Feret diameter” was noted to increase from 1.56±0.15 (for control cells) to 3.11±1.76 (ultrasound-treated cells), indicating a change in cell shape upon ultrasound stimulation. This result is in accordance with the SEM images.

### Example 29

### Cell Seeded Constructs in Bioreactor

Cell Morphology

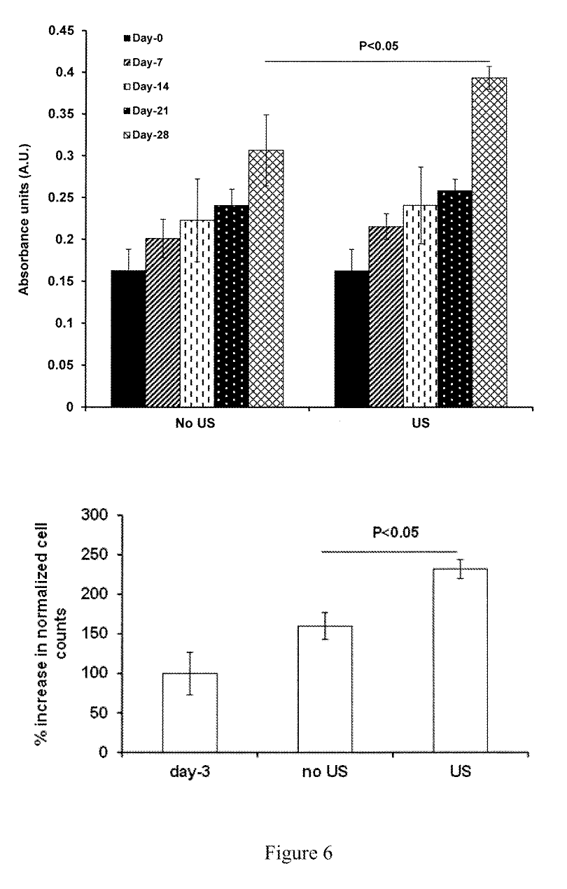

Time-dependent experiments were carried out on cell-seeded scaffolds in the bioreactor. Non-stimulated samples were also maintained in the bioreactor and served as controls. High cellular viabilities were noted at the indicated time-periods, as shown in FIG. 6 (top). A significant difference in cellular viability (p<0.05) was noted between control and ultrasound cellular constructs at day 28. In an independent experiment, plain scaffolds (i.e. without cells; ˜50 scaffolds) were maintained in the bioreactor and the microstructure of the scaffolds was observed to remain intact with no detectable scaffold debris.

By design, the initial seeding density was similar for all the study groups and data was normalized to cell counts obtained at the end of day-3 after cell seeding (2.2×10⁵ cells/scaffold). The effect of ultrasound on the total cell number is shown in FIG. 6 (bottom); a total number of 5.5×10⁵ cells/scaffold were obtained in the ultrasound-treated scaffolds.

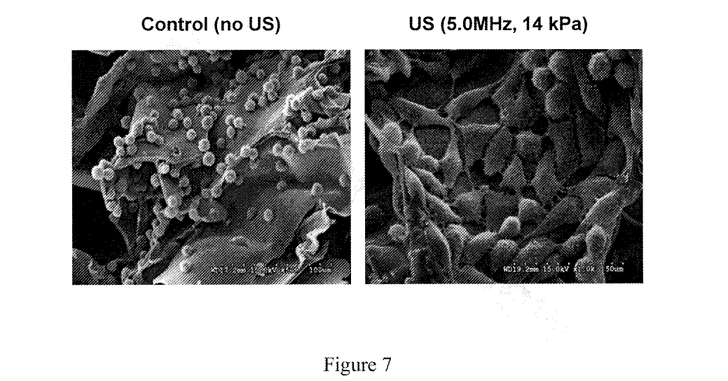

SEM images of ultrasound-stimulated (right image) and control (left image) chondrocytes on scaffolds on day 10 are shown in FIG. 7. The chondrocytes from the control remain spherical and nebulous. However, those subjected to ultrasound treatment show a change in morphology for which the cells start show a spindle-like shape and the presence of long processes.

### Example 30

### Gene Expression of Cartilage-Specific Markers

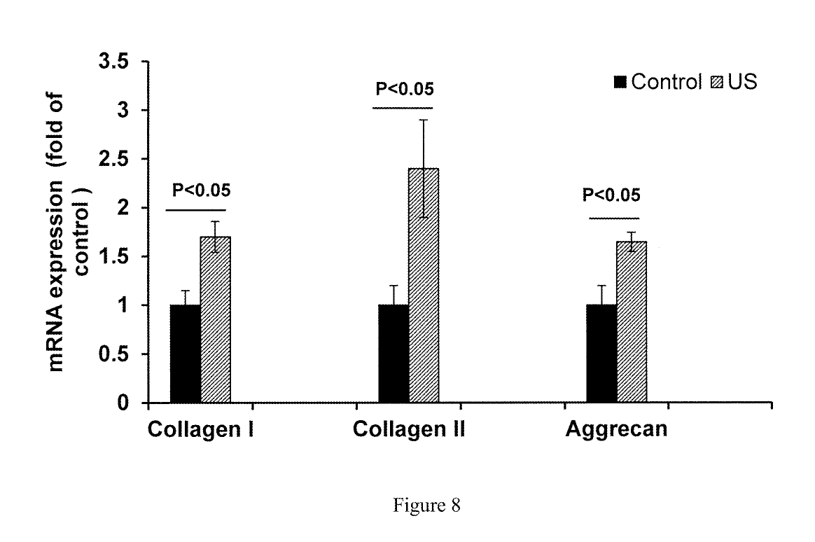

The impact of ultrasound stimulation on the mRNA expression of chondrocytic markers (collagen II, collagen I, aggrecan) was examined by qRT-PCR (FIG. 8). Compared to the control, higher levels of gene expression were noted upon ultrasound.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of culturing cells or tissue, comprising:
exposing cells or tissue in culture to intermittent low-intensity-diffuse ultrasound.

2. The method of claim 1, wherein the intermittent low-intensity-diffuse ultrasound comprises a frequency of from about 1 MHz to about 8 MHz.

3. The method of claim 2, wherein the intermittent low-intensity-diffuse ultrasound comprises a pressure of from about 14 kPa to about 60 kPa.

4. The method of claim 2, wherein the intermittent low-density-diffuse ultrasound comprises a duration of exposure of from about 0.5 min to about 10 mins.

5. The method of claim 2, wherein the intermittent low-density-diffuse ultrasound comprises exposure at an interval of from about 8 times per day up to about 16 times per day.

6. The method of claim 1, wherein the cells are selected from the group consisting of osteoblasts, osteoclasts, osteocytes, chondrocytes, hepatocytes, islet cells, myocytes, epithelial cells, kidney cells, neurons and stem cells.

7. The method of claim 1, wherein the tissue is selected from the group consisting of bone, cartilage, liver, pancreas, muscle, epithelium, kidney, uterus, ovarian, and testes.

8. A method of inducing phosphorylation in cells by extracellular signal-regulated kinases 1 and 2 (Erk1/2), comprising:
exposing cells in culture to intermittent low-intensity-diffuse ultrasound.

9. A method of inducing reorganization of actin in cells, comprising:
exposing cells in culture to intermittent low-intensity-diffuse ultrasound.

10. A bioreactor for culturing cells or tissue, wherein the bioreactor comprises at least one ultrasonic transducer configured to provide an intermittent low-density-diffuse ultrasound to cells or tissues during culture.

11. The bioreactor of claim 10, wherein a tissue culture plate comprising the cells or tissue is in fluid communication with the at least one ultrasonic transducer.

12. The bioreactor of claim 10, wherein the at least one ultrasonic transducer is mounted within a cavity, which is in communication with a tissue culture plate comprising the cells or tissue.

13. The bioreactor of claim 10, wherein the bioreactor comprises at least two ultrasonic transducers configured to provide an intermittent low-density-diffuse ultrasound to the cells or tissues during culture.

14. The bioreactor of claim 13, wherein each of the at least two ultrasonic transducers is configured to deliver different frequencies and/or different pressures of intermittent low-density-diffuse ultrasound to the cells or tissue during culture.

15. The bioreactor of claim 10, further comprising a positioning stage upon which a tissue culture plate is seated, wherein the positioning stage allows for changing the distance between the at least one ultrasound transducer and the cells or tissues comprised within the tissue culture plate.

16. The bioreactor of claim 10, further comprising a microprocessor.

17. The bioreactor of claim 10, wherein the cells are selected from the group consisting of osteoblasts, osteoclasts, osteocytes, chondrocytes, hepatocytes, islet cells, myocytes, epithelial cells, kidney cells, neurons, and stem cells.

18. The bioreactor of claim 10, wherein the tissue is selected from the group consisting of bone, cartilage, liver, pancreas, muscle, epithelium, kidney, uterus, ovarian, and testes.
